(19) United States
(12) Patent Application Publication
(10) Pub. No.: US 2019/0387823 A1
(43) Pub. Date: Dec. 26, 2019

(54) ADAPTIVE CLOTHING

(71) Applicant: O'Brien Capital LLC, San Diego, CA (US)

(72) Inventor: Max O'Brien, San Diego, CA (US)

(21) Appl. No.: 16/449,199

(22) Filed: Jun. 21, 2019

Related U.S. Application Data
(60) Provisional application No. 62/688,621, filed on Jun. 22, 2018.

(51) Int. Cl. A41D 31/18; A61F 2/54; A61F 2/60; A41B 1/10; A41B 7/02; A41D 1/06

(52) U.S. Cl. CPC A41D 31/185; A41D 2400/82; A61F 2/60; A41B 1/10; A41B 7/02; A41D 1/06; A61F 2002/543; A61F 2002/546; A61F 2002/607; A61F 2002/608; A41D 2300/32; A41D 2300/322; A41D 2300/324; A41D 2300/326; A41D 2300/33; A41D 2300/332; A61F 2/54

(57) ABSTRACT

An individual who has had a limb amputation and wears a prosthetic limb on an amputated residual limb may have a difficult time adjusting the prosthetic limb discreetly in a public setting. Described herein are articles of clothing, access opening systems, clothing cuffs, or devices for securing a prosthetic limb to a residual limb fitted with the prosthetic limb and for providing access to the prosthetic limb discreetly without removing a substantial portion of the clothing.

### CROSS-REFERENCE

This application claims the benefit of U.S. Provisional Application No. 62/688,621, filed Jun. 22, 2018, the contents of which are hereby incorporated by reference in their entirety.

### BACKGROUND

Individuals with limb amputations who wear a prosthetic limb or other medical devices can have a difficult time finding clothing that is functional, fashionable, and comfortable to wear. Typical clothing can present a number of issues for its wearer with a prosthetic limb, including bunching up, binding, or drooping of excess fabric in the limb with prosthesis, or difficulty in gaining access to the prosthetic limb to make any adjustments to the prosthetic limb. These issues can cause physical discomfort, decreased performance, mental stress and anguish for the clothing wearer with a prosthetic limb or other medical devices and pose a challenge of finding clothing that fits the wearer with a prosthetic limb or other medical devices and their lifestyle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an example of a pair of pants having access opening assemblies or clothing cuffs on the left pants sleeve above and below the knee.

FIG. 2 shows an example of a shirt having access opening assemblies or clothing cuffs on the left arm sleeve above and below the elbow.

FIG. 3 shows examples of closures and seals for the opening access system.

FIG. 4 shows an example of a shirt having an opening access system or a clothing cuff on the right arm sleeve in a single layer design for a below the elbow amputee.

FIG. 5 shows an example of a shirt having an opening access system or a clothing cuff on the right arm sleeve in a two layer design for a below the elbow amputee.

FIG. 6 shows an example of a shirt having an opening access system or a clothing cuff in a single layer design for an above the elbow amputee.

FIG. 7 shows an example of a shirt having an opening access system or a clothing cuff in a two layer design for an above the elbow amputee.

FIG. 8 shows an example of a pair of pants having an opening access system or a clothing cuff on the left pants sleeve in a single layer design for an above the knee amputee.

FIG. 9 shows an example of a pair of pants having an opening access system or a clothing cuff on the left pants sleeve in a two layer design for an above the knee amputee.

FIG. 10 shows an example of a pair of pants having an opening access system or a clothing cuff on the left pants sleeve in a single layer design for a below the knee amputee.

FIG. 11 shows an example of a pair of pants having an opening access system or a clothing cuff on the left pants sleeve in a two layer design for a below the knee amputee.

FIG. 12 shows an example of a pair of pants having a plurality of opening access systems or clothing cuffs.

FIG. 13 shows an example of a shirt having a plurality of opening access systems or clothing cuffs.

### DETAILED DESCRIPTION

An individual who has had a limb amputation and wears a prosthetic limb on an amputated residual limb or other medical devices may have a difficult time finding articles of clothing that are functional and comfortable to wear. Typical clothing can present a number of issues for its wearer with a prosthetic limb. These issues can include but are not limited to bunching up, binding, or drooping of excess fabric in the limb with prosthesis, or difficulty in gaining access to the prosthetic limb to make any adjustments to the prosthetic limb. As the individual moves, the excess fabric around the prosthetic limb can bunch up and rub against the prosthetic limb, making it more likely for the clothing to develop holes and for the clothing to become damaged. In addition, as the individual moves, connection or a seal between the prosthetic limb and the residual limb can be broken, and the compromised connection or seal can cause discomfort for the individual. The discomfort may be alleviated by adjusting the prosthetic limb. For the individual to adjust the prosthetic limb, the individual may need to remove the article of clothing from a substantial portion of the body to gain access to and adjust the prosthetic limb. Clothing having an access opening system substantially improves wearability for individuals with prosthetic limbs or other medical devices. Individual wearing such clothing having an access opening system can access the prosthetic limb discreetly and in public settings without the inconvenience of removing a substantial portion of the clothing to facilitate adjustment. The clothing having the access opening system also helps in some cases to secure the prosthetic limb on the residual limb and prevent undesired separation of the prosthetic limb from the residual limb. Clothing described herein can also allow wearers having an easy access to the shoulder or hip portions.

Clothing described herein are designed to help users get in and out of clothing easily by providing larger access points, as well as, allow the user to open small sections of the clothing to access their prosthetic without removing clothing. Disclosed herein are access opening systems for accessing a prosthetic limb or other medical devices, such as a prosthetic limb held in place on an individual. The adaptive clothing items, access opening assemblies, clothing cuff, and other items described herein can be applied to prosthetic limbs and other medical devices. Prosthetic limbs are used to exemplify the structure, use and suitability of the items described herein, and it is understood that the adaptive clothing items, access opening assemblies, clothing cuff, and other items described herein may also be applied to wearers of other medical devices.

In some embodiments, the clothing described herein is for an easy access to the shoulder area. In some embodiments, the clothing described herein is for an easy access to the hip area. Use of the devices disclosed herein variously facilitates discreetly accessing or adjusting a prosthetic or other medical devices, such as to secure a prosthetic limb on a residual limb. Some such access opening systems comprises an access opening, a seal comprising a slip-resistant material that can be coupled circumferentially to the inner surface of a pants leg near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. The clothing adapted with the access opening system can provide stability for the prosthetic limb to stay on the residual limb and prevent undesired separation of the prosthetic limb from the residual limb.

Similarly described herein are clothing cuffs for accessing a prosthetic limb or other medical devices discreetly and securing prosthetic limb on a residual limb or other medical devices close to wearer's body. Some such clothing cuffs comprise an interior material, a re-sealable opening that bisects the interior material, and an exterior material that comprises a clothing fabric. Some suitable interior materials have at least one of an elastic deformability and an interior surface having a coefficient of friction that prevents sliding of an object interior to the clothing cuff across the interior surface. The interior material may be a stretchy material that can return to its original dimensions without any force. For example, the interior material may be a slip-resistant material that can help to hold a prosthetic limb in place on the residual limb. The interior material may recoil to a diameter substantially less than a diameter of an adjacent portion of the clothing. Sometimes, the exterior material encloses the interior material. Alternatively, the exterior material may not enclose the interior material. Clothing having such a cuff can secure the prosthetic limb to stay on the residual limb and prevent undesired separation of the prosthetic limb from the residual limb. Such a configuration provides more comfort for the wearers of the clothing having a clothing cuff.

Also described herein are devices for securing a prosthetic limb that comprises a cuff, a re-sealable opening that bisects a band material of the cuff, and a securing strap that tethers the cuff, such as to the torso of a user. Various materials are consistent with the disclosure herein. For example, some cuffs comprise a band material that is elastic and an interior surface having a coefficient of friction that prevents sliding of an object interior to the clothing cuff across the interior surface. Some band material is configured to recoil to a diameter substantially less than a diameter of an adjacent portion of a clothing article. The interior material may be a stretchy material that can return to its original dimensions without any force. The interior material may be a slip-resistant material that can help to hold a prosthetic limb in place on the residual limb.

Additionally, described herein are articles of clothing having at least one access opening system as described herein. The adaptive clothing has an access opening system comprising an access opening, a seal comprising a slip-resistant material that can be coupled circumferentially to the inner surface of the clothing near the access opening, and a closure for the access opening.

As used herein, removing a substantial portion of an article of clothing from a body of an individual may refer to completely removing a prosthetic limb and a residual limb or other medical devices out of the limb portion or the sleeve of the clothing. Removing a substantial portion of an article of clothing may refer to removing the torso portion off the body of the individual wearing the clothing. In one example, an individual removing a substantial portion of a pair of pants may loosen the pants from their waist, move the waist portion of the pants down at least one leg, and remove the prosthetic leg and the residual leg out of at least one of the pants sleeves. In some instances, removing a substantial portion of the pants may allow the individual previously wearing the pants to gain access to their thigh and upper leg and adjust the prosthetic leg as necessary. In another example, an individual removing a substantial portion of a shirt may remove the shirt from their torso and over at least one shoulder, and remove the prosthetic arm and the residual arm out of the shirt sleeve. In some instances, removing a substantial portion of the shirt may allow the individual previously wearing the shirt to gain access to their upper arm and shoulder and adjust the prosthetic arm as necessary.

The article of clothing described herein may comprise a primary material. The primary material may be a flexible fabric. Some flexible fabrics include but are not limited to cotton, polyester, silk, spandex, rayon, acetate, neoprene, fabric made of natural fibers, fabric made of synthetic fibers, or combinations thereof. The flexible fabric may be configured to support a high range of motion of the limb and the prosthetic limb. The flexible fabric may provide increased flexibility and range of motion in areas around the prosthetic limb and functioning natural joints. The flexible fabric can provide a smooth surface where it does not catch on the prosthetic limb.

In some instances, the fabric for the adaptive clothing may have a high breathability and a high air permeability. The breathability of a material may refer to its ability to allow moisture vapor to be transmitted through the material. The air permeability of a material may refers to the pressure difference from the airflow pass through the material. In some instances, the material may have low permeability.

In various embodiments, the material of the adaptive clothing has various thicknesses. In some instances, the thickness of the primary material is at least 0.05 mm, 0.1 mm, 0.2 mm, 0.3 mm, 0.4 mm, 0.5 mm, 0.6 mm, 0.7 mm, 0.8 mm, 0.9 mm, or 1 mm. In some instances, the thickness of the primary material is less than or equal to 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or greater than 10 mm.

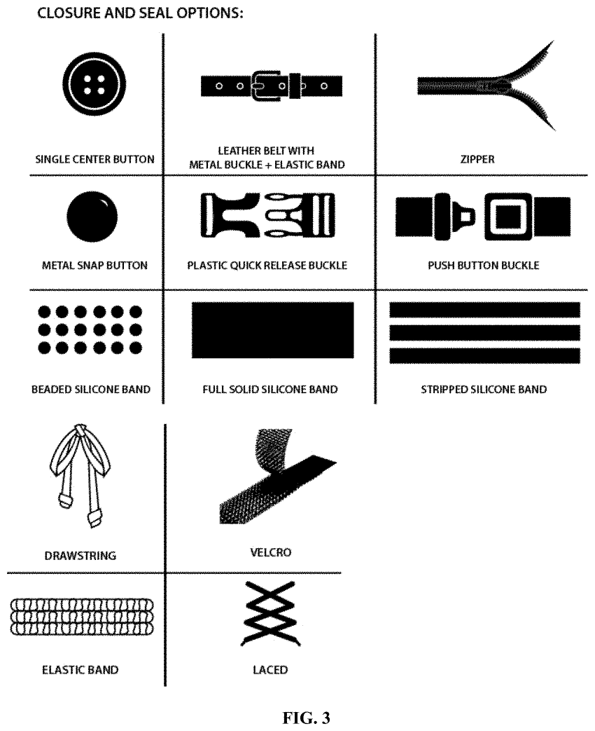

The adjustable closure, also referred as re-sealable opening herein, may be configured to open and close the access opening. The adjustable closure may provide an easy access to the prosthetic limb and allow the individual wearing the article of clothing to remove their prosthetic limb and make adjustments to the prosthetic or residual limb without the inconvenience of removing a substantial portion of their clothing. The closure can be configured to be parallel to a longitudinal axis of the limb. Alternatively or in combination, the closure may be configured to be perpendicular to a longitudinal axis of the limb. Opening of the closure or the re-sealable opening may release tension in the seal, also referred herein as the interior material, when deployed around a prosthetic limb. The closure may include but are not limited to a zipper, a hook and loop fastener (e.g., Velcro), a drawstring, a belt, an elastic band, a plurality of buttons, a plurality of snap closures, a buckle, a magnetic closure, or a combination of a rope and a plurality of holes or hooks. The closure may also include silicone, polymers, loop and tassels, buckles, straps, clips, or snaps. As shown in FIG. 3, examples of the types of materials used for the closure may include single center button, a leather belt with metal buckle and an elastic band, a zipper, a metal snap button, a plastic quick release buckle, a push button buckle, drawstring, Velcro, an elastic band, or a laced tie.

In various embodiments, the closure provides a secure closing of the access opening. The closure strength may be tested in a tensile testing or peel off testing for the chosen closure type. In some instances, ASTM or ISO standard tests are used. In some instances, the closure withstands at least 10 N, 20 N, 30 N, 40 N, 50 N, 60 N, 70 N, 80 N, 90 N, 100 N, 200 N, 300 N, 400 N, 500 N, 600 N, 700 N, 800 N, 900 N, 1000 N, 5000 N, 10000 N, or 50000 N. In some instances, the closure withstands less than or equal to 10 N, 20 N, 30 N, 40 N, 50 N, 60 N, 70 N, 80 N, 90 N, 100 N, 200 N, 300 N, 400 N, 500 N, 600 N, 700 N, 800 N, 900 N, 1000 N, 5000 N, 10000 N, 50000 N, or greater than 50000 N.

The seal, also referred as the interior material or the band material herein, may be chosen to keep the article of clothing fitted close to the prosthetic limb. The seal may keep the article of clothing in its proper orientation, without twisting or bunching up, by adhering to the prosthetic limb with tension or friction. The seal or the interior material may secure a prosthetic limb deployed within the cuff when the closure or the re-sealable opening is sealed. The seal material may have a high coefficient of friction and a high elastic modulus. The seal may comprise a material that provides a sticky or a tacky surface that grips onto a material in direct contact with the seal and provides a resistance in moving the material off the seal. The seal material may comprise rubber, latex, silicone, elastomer, polyurethane, or magnets. As shown in FIG. 3, examples of the types of materials used for the seal may include beaded silicone bands, full solid silicone bands, or stripped silicone bands. In some instances, the seal may comprise a different material from the body portion of the article of clothing. In some instances, the seal may comprise the same material as the body portion of the article of clothing. In some instances, the seal may comprise a different material from the limb portion of the article of clothing. In some instances, the seal may comprise the same material as the limb portion of the article of clothing.

The material for the seal may have a coefficient of friction equal to or greater than about 1.0. In some instances, the material for the seal has a coefficient of friction of at least 0.5, 1.5, 2.0, 2.5, 3.0, 3.5, 4.0, 4.5, 5.0, or greater than 5.0.

The material for the seal may have an elastic modulus equal to or less than about 6 N/mm². In some instances, the material for the seal has an elastic modulus of no greater than 10 N/mm², 9 N/mm², 8 N/mm², 7 N/mm², 5 N/mm², 4 N/mm², 3 N/mm², 2 N/mm², or 1 N/mm² or less.

The seal may comprise a textured surface, where the textured surface provides slip resistance. The material for the seal can be continuous along the circumference of the seal or partially covers the circumference of the seal. The material for the seal may cover at least 10%, 20%, 25%, 30%, 40%, 50%, 60%, 70%, 80%, 90%, 99% or up to 100% of the circumference of the seal. The material for the seal may cover at most 10%, 20%, 25%, 30%, 40%, 50%, 60%, 70%, 80%, 90%, or up to 100% of the circumference of the seal.

The seal may have a width appropriate to support the prosthetic limb in its place and keep the prosthetic limb from moving across its surface. The width of the seal may be at least 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. The width of the seal may be no greater than 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, or greater than 20 cm.

In some instances, the seal may comprise beaded or interspaced materials. The beads comprising the seal may have an end-to-end length or a diameter of at least 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. The beads may have an end-to-end length or a diameter of no greater than 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. The beads may have a center to center distance between two beads of at least 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. The beads may have a center to center distance between two beads of no greater than 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm.

In some instances, the seal may comprise stripped materials. The stripped materials may have at least 2, 3, 4, 5, 6, 7, 8, 9, or 10 strips. The stripped materials may have be less than or equal to 2, 3, 4, 5, 6, 7, 8, 9, or 10 strips. In some instances, the stripped materials is spaced apart at least 0.5 cm, 1 cm, 2 cm, 3 cm, 4 cm, 5 cm, 6 cm, 7 cm, 8 cm, 9 cm, or 10 cm. In some instances, the stripped materials is spaced apart less than or equal to 0.5 cm, 1 cm, 2 cm, 3 cm, 4 cm, 5 cm, 6 cm, 7 cm, 8 cm, 9 cm, or 10 cm. In some instances, the width of the strip is at least 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. In some instances, the width of the strip is no greater than 0.5 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm. In some instances, the strips are interspaced or beaded. In some instances, the strips are continuous.

In various embodiments, the seal may have various thicknesses. The seal may have a low profile or a low thickness. In some instances, the thickness of the seal is at least 0.05 mm, 0.1 mm, 0.2 mm, 0.3 mm, 0.4 mm, 0.5 mm, 0.6 mm, 0.7 mm, 0.8 mm, 0.9 mm, 1 mm or greater than 1 mm. In some instances, the thickness of the seal is no greater than 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, or 10 mm, or greater than 10 mm.

In some instances, a durable portion may be incorporated to the adaptive clothing described herein. The durable portion may comprise a resilient material to withstand the repeated abrasion from the rigid material of the prosthetic limb or other medical devices worn by the individual wearing the clothing. The durable portion may be placed in the areas that are subject to the repeated abrasion. The durable portion may incorporate a high performance fiber on the inside or the outside of the limb portion of the clothing. The fabric can provide a comfortable protective layer to reduce damages to the clothing from abrasion between the rigid surfaces of the prosthetic limb or other medical devices and the clothing.

The durable portion may include but are not limited to para-aramid synthetic fiber (e.g., Kevlar), high tensile fabrics, or fabrics with special weaves or high density weaves to provide a high tensile strength. The durable portion may comprise para-aramid synthetic materials such as Kevlar, Twaron; poly (phenylene benzobizoxazole) (PBO) fibers; ultra-high molecular weight polyethylene (UHMWPE) materials such as Dyneema, Spectra, and Spectra Shield; polyhydroquinone-diimidazopyridine (PIPD) fibers such as M5, Vectram (Hoechst Calaneses), Technora (Tejin) and Nextel (3M); and laminated cross-laid non-woven fibers in a resin matrix such as GoldFlex. These materials may have a high stiffness and strength to weight ratio.

The durable portion may comprise a material with a high abrasive strength. The durable material may be capable of withstanding at least 5000 cycles in an abrasion test. The abrasion test may be ASTM D4966, Standard Test Method for Abrasion Resistance of Textile Fabrics (Martindale Abrasion Test Method). In some instances, the durable material may be capable of withstanding at least 1000 cycles, 2000 cycles, 3000 cycles, 4000 cycles, 5000 cycles, 6000 cycles, 7000 cycles, 8000 cycles, 9000 cycles, 10000 cycles, or greater than 10000 cycles in an abrasion test.

The fabric for the durable portion may comprise a high-performance fiber having a tensile strength of at least 5 grams/denier. In some instances, the fabric may have a tensile strength of at least 1 gram/denier, 2 grams/denier, 3 grams/denier, 4 grams/denier, 5 grams/denier, 6 grams/denier, 7 grams/denier, 8 grams/denier, 9 grams/denier, 10 grams/denier, or 20 grams/denier. Denier is defined as a unit of measurement used to determine the fiber thickness of individual threads or filaments used in the creation of textiles and fabrics. Fabrics with a high denier count tend to be thick, sturdy, and durable while fabrics with a low denier count tend to be sheer, soft, and silky.

In various embodiments, the durable material may have various thicknesses. The durable material may be thicker than the primary fabric for the adaptive clothing. In some instances, the thickness of the durable material may be at least 0.05 mm, 0.1 mm, 0.2 mm, 0.3 mm, 0.4 mm, 0.5 mm, 0.6 mm, 0.7 mm, 0.8 mm, 0.9 mm, 1 mm, or greater than 1 mm. In some instances, the thickness of the durable material may be no greater than 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, or 10 mm, or more than 10 mm.

The inner layer of the clothing may be configured to fit around the outer surface of the proximal end portion of the prosthetic limb and the limb. The inner layer may help secure and support the prosthetic limb in its proper position during movement or at rest. The inner layer may be configured to secure the prosthetic limb from rotating around a longitudinal axis of the limb or translating off the axis. The inner layer may be coupled to the first limb portion of the article or be removable from the first limb portion of the article. The inner layer may comprise a magnet configured to align with a closure or a seal comprising a magnet.

The inner layer may comprise a fabric with a high elasticity. The inner layer may comprise spandex, elastane, nylon, polyester, silk, wood fiber fabric, or rayon. The inner layer material may have an ultimate tensile strain of at least 2. In some instances, the inner layer material may have an ultimate tensile strain (length/length) of at least 1, 2, 3, 4, 5, 6, 7, 8, 9, or 10, or greater than 10.

Sometimes the clothing described herein has a pocket on the body portion or the first limb portion. The pocket may have an inner surface comprising a waterproof material. The inner surface material may include but are not limited to silicone, polymers, waterproof materials, or materials with a high surface tension. The inner surface material for the pocket may have low breathability. In some instances, the inner surface material may have a waterproof rating of greater than or equal to 5000 mm. The pocket may comprise a pocket closure, where the closure may include but is not limited to a zipper, a hook and loop fastener (Velcro), a drawstring, a belt, an elastic band, a plurality of buttons, a plurality of snap closures, a buckle, a magnetic closure, or a combination of a rope and a plurality of holes or hooks. The pocket may provide a specialized storage, such as holding prosthetic accessories. In one example, the pocket may be lined with a waterproof material to provide a waterproof closure and a dry environment to store batteries or accessories for a prosthetic limb or other medical devices.

An individual may wear the articles of clothing, access opening systems, clothing cuffs, or devices that place the access opening at or near a proximal portion, or a socket, of the prosthetic limb that fits into the residual limb or other medical devices when the articles of clothing, access opening systems, clothing cuffs, or devices is worn by the individual. The devices described herein may be worn by an individual wearing at least one prosthetic limb to secure and provide access to at least one prosthetic limb. The devices described herein may secure and provide access to a prosthetic limb worn by the individual who had an amputation at above the knee, below the knee, above the elbow, or below the elbow and is wearing the prosthetic limb on the residual limb. The devices described herein may be worn by an individual to secure and provide access to a prosthetic limb for an amputation of one limb, or to multiple prosthetic limbs worn by the individual. In some instances, the individual may have had amputations to both legs, where both legs amputations are at above the knee, or both leg amputations are at below the knee, or one leg amputation at above the knee and another leg amputation at below the knee. In some instances, the individual may have had amputations to both arms, where both arm amputations are at above the elbow, or both arm amputations are at below the elbow, or one arm amputation at above the elbow and another arm amputation at below the elbow.

The adaptive clothing may have one limb portion having an access opening system, a clothing cuff, or a device for securing a prosthetic limb described herein. In some instances, an article of clothing may have one limb portion or sleeve having the device and another limb portion or sleeve not having the device. In some examples, an adaptive pants may have the devices located at above the knee or below the knee of the individual wearing the adaptive pants. In some examples, an adaptive pants may have the devices located at middle of the femur or the upper leg of the individual wearing the adaptive pants. In other examples, an adaptive pants may have the devices located at middle of the tibia or the lower leg of the individual wearing the adaptive pants. For example, an adaptive shirt may have the device at above the elbow or below the elbow on the individual wearing the adaptive shirt. In some examples, an adaptive shirt may have the device at middle of the humerus or upper arm of the individual wearing the adaptive shirt. In other examples, an adaptive shirt may have the device at middle of the ulna or radius or the lower arm of the individual wearing the adaptive shirt.

The adaptive clothing may have multiple limb portions having access opening systems, clothing cuffs, or devices described herein. The clothing may have two limb portions with the same access opening systems, clothing cuffs, or devices to provide access openings. In some instances, an article of clothing may have two devices, one device on each limb portion or sleeves. The location of the devices along the limb portion or the sleeve may be symmetric or asymmetric on the two limb portions. In some examples, an adaptive pants may have the devices located at the same distance from the waist, one device on each pants sleeve. In other examples, an adaptive pants may have the devices located at the different distance from the waist, one device on one sleeve at above the knee and another device at below the knee individual wearing the adaptive pants. For example, an adaptive shirt may have the devices located at the same distance from the shoulder, one device on each sleeve. In other examples, an adaptive shirt may have the devices located at different distances from the shoulder, one device on one sleeve at above the elbow and another device at below the elbow on the individual wearing the adaptive shirt.

In some instances, the clothing cuff may be configured for a below-knee pant leg opening. Alternatively or in combination, the clothing cuff may be configured for an above-knee pant leg opening. In some instances, the clothing cuff may be configured for a below elbow shirt opening. Alternatively or in combination, the clothing cuff may be configured for an above elbow shirt opening.

In some instances, the device for securing the prosthetic limb may be configured to secure an above-elbow prosthetic, a below-elbow prosthetic, an above-knee prosthetic, a below-elbow prosthetic, or combinations thereof.

In some instances, the article of clothing comprises a clothing cuff described herein and a second cuff not comprising the interior material.

The distal end of the limb portion or the sleeve of the adaptive clothing can be at a knee, an elbow, a wrist, and an ankle of the individual wearing the clothing. In some instances, the distal end of the sleeve may extend distally beyond the access opening, where the access opening is in the middle of the sleeve.

The access opening assemblies, clothing cuffs, or devices for securing prosthetic limbs described herein may be incorporated to articles of clothing by typical methods for preparing an article of clothing. The access opening assembly may be incorporated by sewing, bonding, taping, gluing, or other common methods of incorporating pieces of fabric together. Articles of clothing may be adapted to incorporate the access opening assembly and then may be used as adaptive clothing.

The access opening assemblies, clothing cuffs, or devices for securing prosthetic limbs described herein may be incorporated on the limb portion or the sleeve facing outward away from the center of the body. The outward positioning of the assembly, cuff, or device may provide an easier access point to open and close the access opening and to reach the prosthetic limb. Alternatively or in combination, the opening assemblies, clothing cuffs, or devices may be incorporated on the limb portion or the sleeve facing inward toward the center of the body. The inward positioning of the assembly, cuff, or device may provide a more discreet position for the assembly. The inward positioning may be helpful for more business or formal clothing to make the assembly less noticeable. The closure or re-sealable opening of the assembly may be incorporated along a seam of the article of clothing. The closure or re-sealable opening of the assembly may be incorporated in the middle of the limb portion or the sleeve of the article of clothing. In some instances, the assembly may be covered with an outer layer of clothing. In other instances, the assembly may be on the outer layer of clothing.

Table 1 provides lists of examples of materials for the clothing. The clothing, the torso portion, and the limb portions comprise Type A materials. The durable portion comprises Type B materials. Type C materials may be used for the inner sleeve. The closures and seals may comprise Type D materials. The pocket for the adaptive clothing may comprise Type E material.

TABLE 1
Examples of materials for the adaptive clothing

| Material | | Examples of Materials with Similar |
|---|---|---|
| Reference | Desired Material | Properties (list not exclusive of |
| Label | Properties | other existing types of materials) |
| Type A | Flexible | Spandex, elastic, neoprene, cotton |
| Type B | Durable | Kevlar, high-tensile fabrics; fabrics |
| | | with specialty weaves |
| Type C | Supportive | Elastic fabrics with limited end range; |
| | | compression fabric, fabrics with specialty |
| | | weaves |
| Type D | Seals & Closures | Silicone, polymers, buttons, zippers, |
| | | drawstring, loop & eltassel, velcro, |
| | | buckles, straps, clips, snaps, magnets |
| Type E | Specialized | Silicone, polymers, zippers, velcro |
| | Storage | |

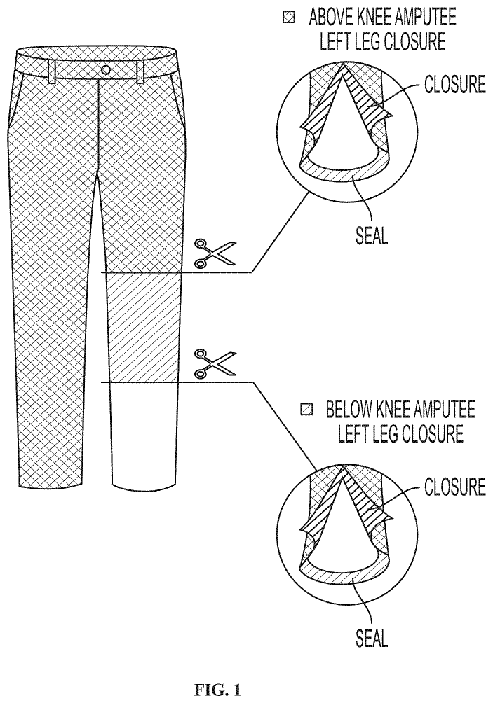

FIG. 1 shows an example of a pair of adaptive pants having access opening assemblies, clothing cuffs, or devices for securing prosthetic limbs as described herein. The assemblies are shown on the left pants sleeve at above the knee and also at below the knee on an individual wearing the adaptive pants. An assembly comprises a closure, which is shown as a vertical, overlapping, re-sealable opening, and a seal, which is shown as a continuous band around the inner circumference of the sleeve, in FIG. 1. The seal may be found at the distal end of the sleeve as shown in FIG. 1 or in the middle of the sleeve.

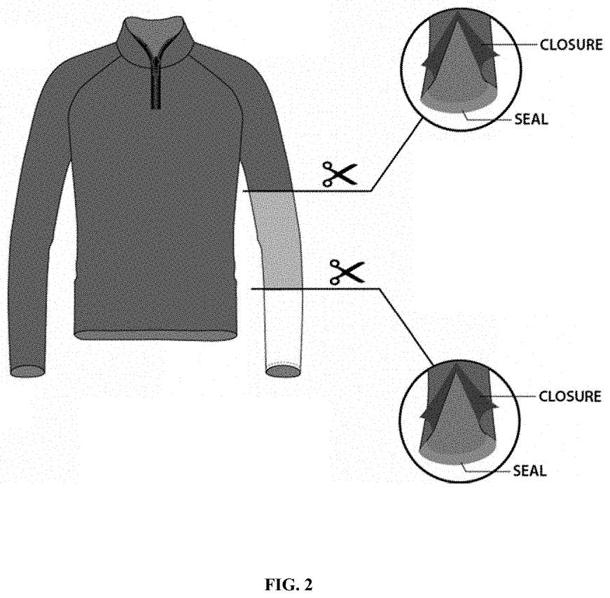

FIG. 2 shows an example of an adaptive shirt having access opening assemblies, clothing cuffs, or devices for securing prosthetic limbs. The assemblies are shown on the left arm sleeve at above and below the elbow on an individual wearing the adaptive shirt. An assembly comprises a closure, which is shown as a vertical, overlapping, re-sealable opening, and a seal, which is shown as a continuous band around the inner circumference of the sleeve, in FIG. 2. The seal may be found at the distal end of the sleeve as shown in FIG. 2 or in the middle of the sleeve.

FIG. 3 shows examples of closures and seals for the opening access system. Examples of closures include but are not limited to buttons, belts with a buckle and an elastic band, zippers, metal snap buttons, quick release buckles, push button buckles, drawstrings, Velcro, elastic bands, magnetic closures, and laced closures. Examples of seals include but are not limited to beaded silicone bands, full solid silicone bands, stripped silicone bands, or elastic bands.

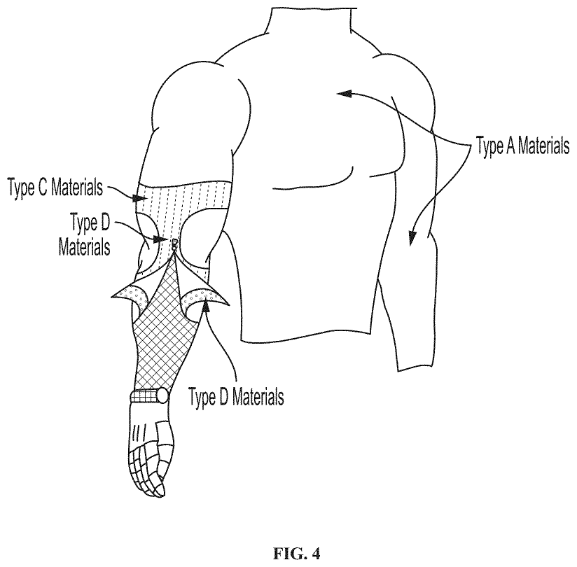

FIGS. 4-13 show various embodiments in which the access opening systems, clothing cuffs, or devices described herein may be secured to the prosthetic limb and may be integrated into articles of clothing or used with other articles of clothing.

FIG. 4 shows an example of a shirt having an opening access system or a clothing cuff on the right arm sleeve in a single layer design for a below the elbow amputee. The adaptive shirt comprises an outer shell and an access opening system having a seal to secure the socket of the prosthetic arm. The seal and the closure can be provided in a single layer. The outer shell comprises a material that is flexible, elastic, or breathable or a type A material from Table 1. The outer shell of the shirt comprises a material that is typically found in clothing and incorporates the amputee arm sleeve. The sleeve material adjacent to the opening access system comprises a material that is supportive or durable or a type C material from Table 1. The sleeve material adjacent to the opening access system can provide a secondary suspension. In some instances, the material of the closure system is a zipper or a type D material from Table 1. In some instances, material for the seal system is silicone beads or elastic bands or a type D material from Table 1.

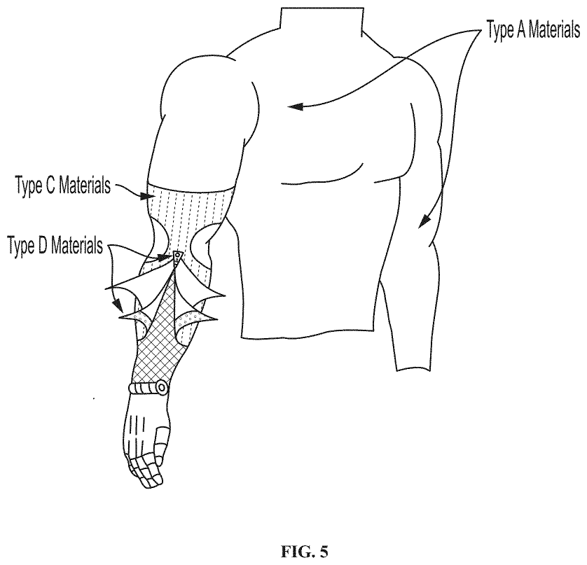

FIG. 5 shows an example of a shirt having an opening access system or a clothing cuff on the right arm sleeve in a two layer design for a below the elbow amputee. The seal can be provided in an inner layer of the opening access system, and the closure can be provided in both layers of the opening access system. The outer shell comprises a material that is flexible, elastic, or breathable or a type A material from Table 1. The outer shell of the shirt comprises a material that is typically found in clothing and incorporates the amputee arm sleeve. The sleeve material adjacent to the opening access system comprises a material that is supportive or durable or a type C material from Table 1. The sleeve material adjacent to the opening access system can provide a secondary suspension. In some instances, the material of the closure system is a zipper or a type D material from Table 1. In some instances, material for the seal system is silicone beads or elastic bands or a type D material from Table 1.

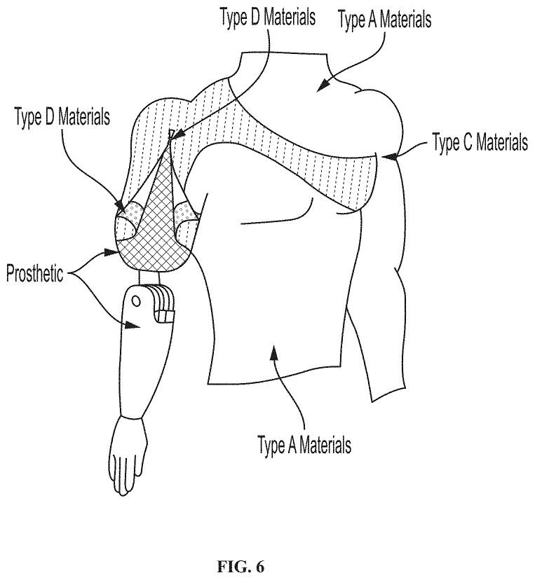

FIG. 6 shows an example of a shirt having an opening access system or a clothing cuff in a single layer design for an above the elbow amputee. The seal and the closure can be provided in a single layer. The outer shell comprises a material that is flexible, elastic, or breathable or a type A material from Table 1. The outer shell of the shirt comprises a material that is typically found in clothing and incorporates the amputee arm sleeve. The opening access system can be incorporated into amputee arm sleeve that comprises a suspender running across from the shoulder of the residual arm to the underarm area on the side opposite the residual limb of a wearer. The amputee sleeve material comprises a material that is supportive or durable or a type C material from Table 1. The amputee sleeve material can provide a secondary suspension. In some instances, the material of the closure system is a zipper or a type D material from Table 1. In some instances, material for the seal system is silicone beads or elastic bands or a type D material from Table 1.

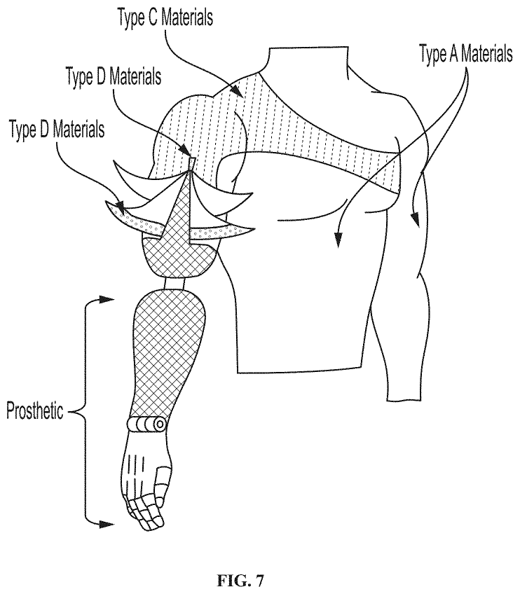

FIG. 7 shows an example of a shirt having an opening access system or a clothing cuff in a two layer design for an above the elbow amputee. The seal can be provided in an inner layer of the opening access system, and the closure can be provided in both layers of the opening access system. The outer shell comprises a material that is flexible, elastic, or breathable or a type A material from Table 1. The outer shell of the shirt comprises a material that is typically found in clothing and incorporates the amputee arm sleeve. The opening access system can be incorporated into amputee arm sleeve that comprises a suspender running across from the shoulder of the residual arm to the underarm area on the side opposite the residual limb of a wearer. The amputee sleeve material comprises a material that is supportive or durable or a type C material from Table 1. The amputee sleeve material can provide a secondary suspension. In some instances, the material of the closure system is a zipper or a type D material from Table 1. In some instances, material for the seal system is silicone beads or elastic bands or a type D material from Table 1.

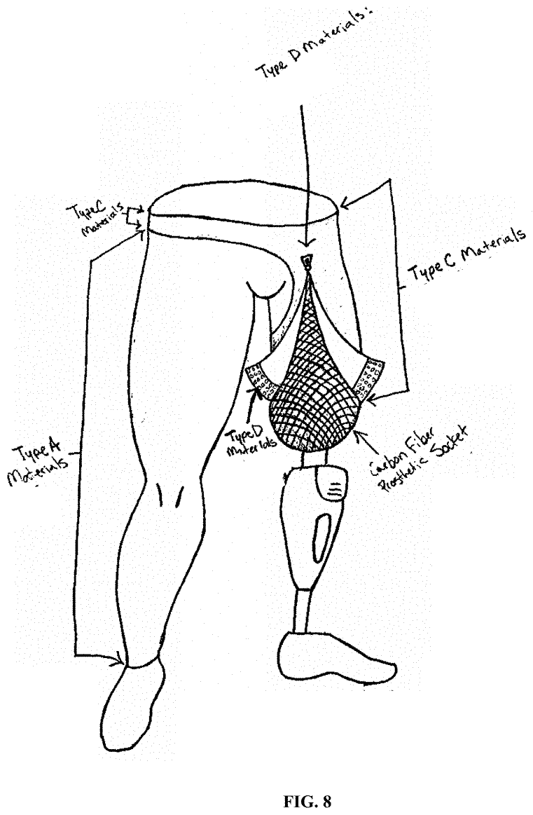

FIG. 8 shows an example of a pair of pants having an opening access system or a clothing cuff on the left pants sleeve in a single layer design for an above the knee amputee. The seal and the closure can be provided in a single layer. The outer shell comprises a material that is flexible, elastic, or breathable or a type A material from Table 1. The outer shell of the pants comprises a material that is typically found in clothing and incorporates the adaptive pants sleeve. The opening access system can be incorporated into adaptive pants sleeve that comprises a suspender running along waist and extending down the residual limb of a wearer. The adaptive pants sleeve material comprises a material that is supportive or durable or a type C material from Table 1. The amputee sleeve material can provide a secondary suspension. In some instances, the material of the closure system is a zipper or a type D material from Table 1. In some instances, material for the seal system is silicone beads or elastic bands or a type D material from Table 1.

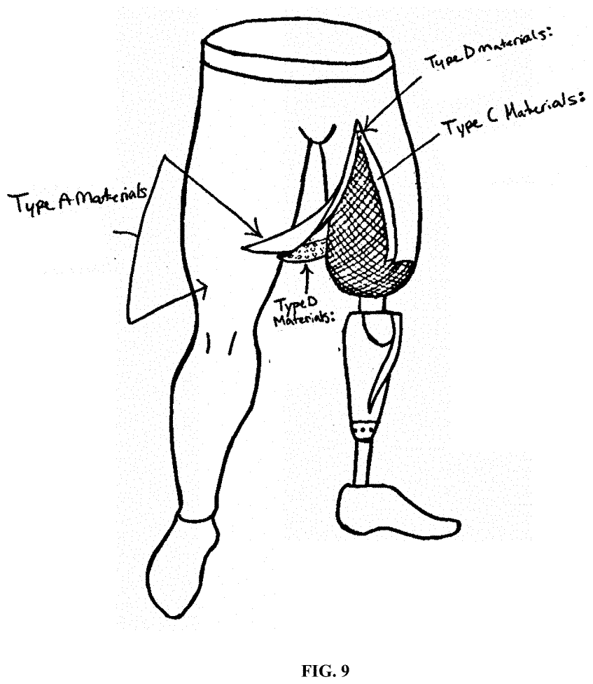

FIG. 9 shows an example of a pair of pants having an opening access system or a clothing cuff on the left pants sleeve in a two layer design for an above the knee amputee. The seal can be provided in an inner layer of the opening access system, and the closure can be provided in both layers of the opening access system. The outer shell comprises a material that is flexible, elastic, or breathable or a type A material from Table 1. The outer shell of the pants comprises a material that is typically found in clothing and incorporates the adaptive pants sleeve. The opening access system can be incorporated into adaptive pants sleeve that comprises a suspender running along waist and extending down the residual limb of a wearer. The adaptive pants sleeve material comprises a material that is supportive or durable or a type C material from Table 1. The amputee sleeve material can provide a secondary suspension. In some instances, the material of the closure system is a zipper or a type D material from Table 1. In some instances, material for the seal system is silicone beads or elastic bands or a type D material from Table 1.

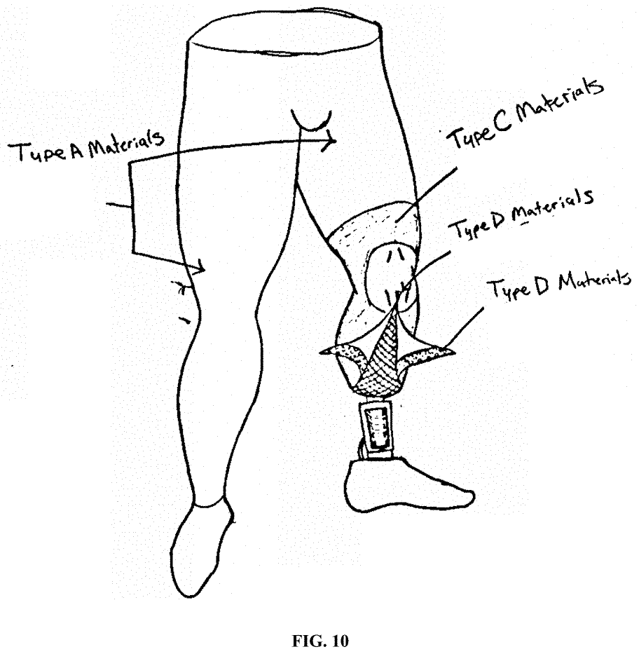

FIG. 10 shows an example of a pair of pants having an opening access system or a clothing cuff on the left pants sleeve in a single layer design for a below the knee amputee. The seal and the closure can be provided in a single layer. The outer shell comprises a material that is flexible, elastic, or breathable or a type A material from Table 1. The outer shell of the pants comprises a material that is typically found in clothing and incorporates the adaptive pants sleeve. The opening access system can be incorporated into adaptive pants sleeve that comprises a suspender running along waist and extending down the residual limb of a wearer. The adaptive pants sleeve material comprises a material that is supportive or durable or a type C material from Table 1. The amputee sleeve material can provide a secondary suspension. In some instances, the material of the closure system is a zipper or a type D material from Table 1. In some instances, material for the seal system is silicone beads or elastic bands or a type D material from Table 1.

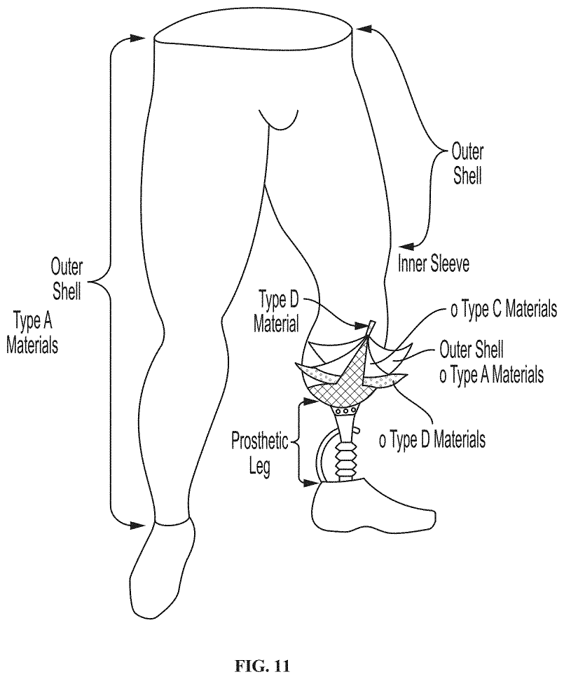

FIG. 11 shows an example of a pair of pants having an opening access system or a clothing cuff on the left pants sleeve in a two layer design for a below the knee amputee. The seal can be provided in an inner layer of the opening access system, and the closure can be provided in both layers of the opening access system. The outer shell comprises a material that is flexible, elastic, or breathable or a type A material from Table 1. The outer shell of the pants comprises a material that is typically found in clothing and incorporates the adaptive pants sleeve. The opening access system can be incorporated into adaptive pants sleeve that comprises a suspender running along waist and extending down the residual limb of a wearer. The adaptive pants sleeve material comprises a material that is supportive or durable or a type C material from Table 1. The amputee sleeve material can provide a secondary suspension. In some instances, the material of the closure system is a zipper or a type D material from Table 1. In some instances, material for the seal system is silicone beads or elastic bands or a type D material from Table 1.

The opening system comprising a seal and a closure are designed to help the user get in and out of clothing easily by providing larger access points. The opening system comprising a seal and a closure allow the user to open small sections of the clothing to access their prosthetic without removing the clothing.

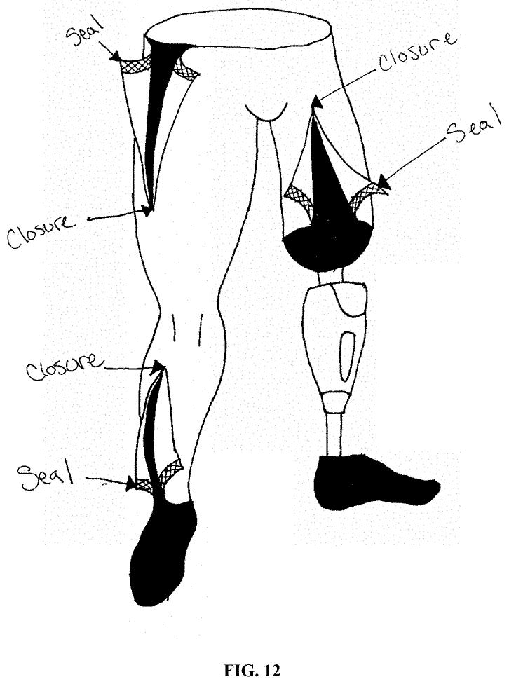

FIG. 12 shows an example of a pair of pants having a plurality of opening access systems or clothing cuffs. The opening access system comprising a seal and a closure can be provided at a number of different places on the adaptive pants. The opening access system comprising a seal and a closure can be provided at multiple places on a pair of pants. The opening system can be placed at the waist vertically on the adaptive pants when the pair of adaptive pants is worn by the user. The opening system can be placed at the distal end of a pants sleeve. The opening system can be placed above the knee vertically along the length of a pants sleeve when the pair of adaptive pants is worn by the user. The opening system can be placed below the knee vertically along the length of a pants sleeve when the pair of adaptive pants is worn by the user. The opening system can be placed at or near the ankle vertically along the length of a pants sleeve when the pair of adaptive pants is worn by the user.

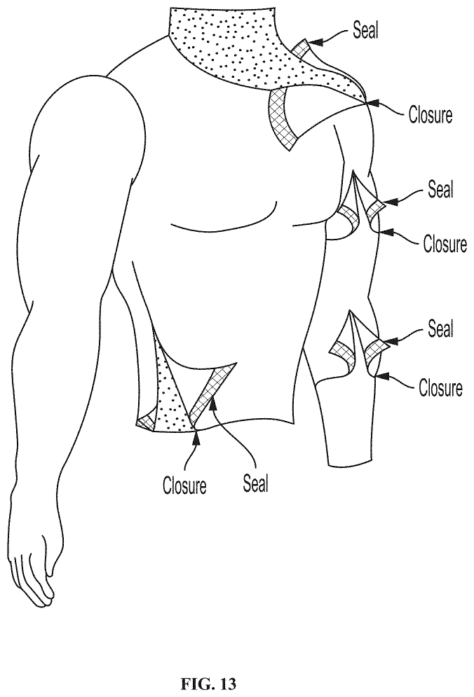

FIG. 13 shows an example of a shirt having a plurality of opening access systems or clothing cuffs. The opening access system comprising a seal and a closure can be provided at multiple places on the adaptive shirt. The opening system can be placed near the neck along the shoulder when the adaptive shirt is worn by the user. The opening system can be placed vertically on body portion of the shirt when the adaptive shirt is worn by the user. The opening system can be placed at the distal end of a shirt sleeve. The opening system can be placed above the elbow vertically along the length of a shirt sleeve when the adaptive shirt is worn by the user. The opening system can be placed below the elbow vertically along the length of a shirt sleeve when the adaptive shirt is worn by the user. The opening system can be placed at or near the wrist along the length of the shirt sleeve when the adaptive shirt is worn by the user.

A variety of the materials can be assembled to produce the adaptive clothing described herein. Sometimes a type B material is used in areas of high stress such as seams around the seals and closures or areas around the prosthetic socket. In some cases, a type E material is incorporated into the adaptive clothing.

### Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein the terms “individual,” “subject,” and “wearer” are used interchangeably and include any member of the animal kingdom, including humans.

### Exemplary Embodiments

Among the exemplary embodiments are:

1. Articles of clothing comprising: a. a body portion configured to at least partially surround a torso of an individual; b. a first limb portion having an outer surface and an inner surface, and a proximal end portion connected to the body portion and a distal end portion having a first opening at a distal end and configured to at least partially surround a limb of the individual; c. an access opening on the distal end portion proximal to the first opening; d. a seal coupled circumferentially to the inner surface at a distal end portion of the access opening; and e. a closure coupled to edge portions of the access opening, wherein a distal end of the closure meets or disrupts a circumference of the seal and wherein the closure is configured to open and close the access opening and configured to adjust the circumference of the seal by closing the closure to adjust an overlapping area of the edge portions of the access opening. 2. The article of clothing of embodiment 1, wherein the access opening is configured to be located at one of above a knee, below the knee, above an elbow, and below the elbow when the article is worn by the individual. 3. The article of clothing of embodiment 1, wherein the seal is configured to fit around and form a tight seal to an outer surface of a proximal end portion of a prosthetic limb when the prosthetic limb is fitted to the limb of the individual. 4. The article of clothing of embodiment 3, wherein the seal is configured to secure the prosthetic limb from rotating around a longitudinal axis of the limb or translating off the axis. 5. The article of clothing of embodiment 3, wherein the access opening is configured to provide access to the prosthetic limb without removing a substantial portion of the article from the individual by opening the closure. 6. The article of clothing of embodiment 3, wherein the access opening is configured to provide access to the prosthetic limb without removing the body portion from the individual by opening the closure. 7. The article of clothing of embodiment 1, wherein the article comprises a flexible fabric. 8. The article of clothing of embodiment 1, wherein the article comprises cotton, polyester, silk, or spandex. 9. The article of clothing of embodiment 1, wherein the flexible fabric is configured to support a high range of motion of the limb and of the prosthetic limb and does not catch on the prosthetic limb. 10. The article of clothing of embodiment 1, wherein the closure is configured to close the access opening. 11. The article of clothing of embodiment 10, wherein the closure comprises a zipper, a hook and loop fastener (Velcro), a drawstring, a belt, an elastic band, a plurality of buttons, a plurality of snap closures, a buckle, a magnetic closure, or a combination of a rope and a plurality of holes or hooks. 12. The article of clothing of embodiment 10, wherein the closure is configured to be parallel to a longitudinal axis of the limb. 13. The article of clothing of embodiment 10, wherein the closure is configured to be perpendicular to a longitudinal axis of the limb. 14. The article of clothing of embodiment 1, wherein the seal comprises a slip resistant material. 15. The article of clothing of embodiment 1, wherein the seal comprises a material with a high coefficient of friction and a high elastic modulus. 16. The article of clothing of embodiment 14, wherein the material has a coefficient of friction equal to or greater than about 1.0. 17. The article of clothing of embodiment 14, wherein the material has an elastic modulus equal to or less than about 6 N/mm². 18. The article of clothing of embodiment 14, wherein the seal comprises a textured surface, wherein the textured surface provides slip resistance. 19. The article of clothing of embodiment 14, wherein the seal comprises rubber, latex, silicone, elastomer, or polyurethane. 20. The article of clothing of embodiment 14, wherein the material is continuous along the circumference of the seal. 21. The article of clothing of embodiment 14, wherein the material partially covers the circumference of the seal. 22. The article of clothing of embodiment 3, wherein the first limb portion comprises a durable portion, wherein the durable portion is coupled to the first limb portion near the access opening and covers at least a portion of the prosthetic limb. 23. The article of clothing of embodiment 22, wherein the durable portion comprises a material with a high abrasive strength. 24. The article of clothing of embodiment 23, wherein the material is capable of withstanding at least 5000 cycles in an abrasion test. 25. The article of clothing of embodiment 22, wherein the durable portion comprises para-aramid synthetic fiber. 26. The article of clothing of embodiment 3, wherein the article further comprises an inner layer, wherein the inner layer is configured to fit around the outer surface of the proximal end portion of the prosthetic limb and the limb. 27. The article of clothing of embodiment 26, wherein the inner layer comprises a fabric with a high elasticity. 28. The article of clothing of embodiment 26, wherein the material has an ultimate tensile strain of at least 2. 29. The article of clothing of embodiment 26, wherein the inner layer comprises spandex, elastane, nylon, polyester, silk, wood fiber fabric, or rayon. 30. The article of clothing of embodiment 26, wherein the inner layer is configured to secure the prosthetic limb from rotating around a longitudinal axis of the limb or translating off the axis. 31. The article of clothing of embodiment 26, wherein the inner layer is coupled to the first limb portion of the article. 32. The article of clothing of embodiment 26, wherein the inner layer is configured to be removable from the first limb portion of the article. 33. The article of clothing of embodiment 1, wherein the article comprises a second limb portion, the second limb portion having an second outer surface and an inner surface, and a proximal end portion connected to the body portion and a distal end portion having a second opening at a distal end and configured to at least partially surround a second limb of the individual. 34. The article of clothing of embodiment 31, wherein the second limb portion comprises a. a second access opening on the distal end portion proximal to the second opening; b. a second seal coupled circumferentially to the inner surface at a distal end portion of the second access opening; and c. a second closure at edge portions of the second access opening, wherein a distal end of the closure meets or disrupts a circumference of the second seal and wherein the closure is configured to open and close the access opening and configured to adjust the circumference of the second seal by closing the closure to adjust an overlapping area of the edge portions of the second access opening. 35. The article of clothing of embodiment 31, wherein the second seal comprises a slip resistant material. 36. The article of clothing of embodiment 31, wherein the second limb portion is same length as the first limb portion. 37. The article of clothing of embodiment 35, wherein the second limb portion comprises a. a second access opening on the distal end portion proximal to the second opening; b. a second seal coupled circumferentially to the inner surface at a distal end portion of the second access opening; and c. a second closure at edge portions of the second access opening, wherein a distal end of the closure meets or disrupts a circumference of the second seal and wherein the closure is configured to open and close the access opening and configured to adjust the circumference of the second seal by closing the closure to adjust an overlapping area of the edge portions of the second access opening. 38. The article of clothing of embodiment 35, wherein the second seal comprises a slip resistant material. 39. The article of clothing of embodiment 1, wherein the distal end of the first limb portion is configured to be located near one of a knee, an elbow, a wrist, and an ankle. 40. The article of clothing of embodiment 1, wherein the distal end of the first limb portion extends distally further than the access opening. 41. The article of clothing of embodiment 1, wherein the article further comprises a pocket on the body portion or the first limb portion. 42. The article of clothing of embodiment 41, wherein the pocket has an inner surface, wherein the inner surface comprises a waterproof material. 43. The article of clothing of embodiment 41, wherein the pocket comprises a pocket closure, wherein the pocket closure comprises a zipper, a hook and loop fastener (Velcro), a drawstring, a belt, an elastic band, a plurality of buttons, a plurality of snap closures, a buckle, a magnetic closure, or a combination of a rope and a plurality of holes or hooks.

44. Access opening assemblies on an article of clothing, the access opening assembly comprising: a. an access opening on a distal end portion of a limb portion of an article of clothing and proximal to a distal opening of the limb portion; b. a seal coupled circumferentially to an inner surface of the limb portion at a distal end portion of the access opening; and c. a closure at edge portions of the access opening, wherein a distal end of the closure meets or disrupts a circumference of the seal and wherein the closure is the closure is configured to open and close the access opening and configured to adjust the circumference of the seal by closing the closure to adjust an overlapping area of the edge portions of the access opening. 45. The access opening assembly of embodiment 44, wherein the seal comprises a slip resistant material.

46. Clothing cuffs comprising a. an interior material having an elastic deformability and an interior surface having a coefficient of friction that does not support sliding of an object interior to the clothing cuff across the interior surface; wherein the interior material is configured to recoil to a diameter substantially less than a diameter of an adjacent portion of a clothing article; b. a re-sealable opening that bisects the interior material; and c. an exterior material comprising a clothing fabric. 47. The clothing cuff of embodiment 45, wherein the exterior material encloses the interior material. 48. The clothing cuff of embodiment 45, wherein the exterior material does not enclose the interior material. 49. The clothing cuff of embodiment 45, wherein the re-sealable opening comprises a zipper. 50. The clothing cuff of embodiment 45, wherein the re-sealable opening comprises a snap. 51. The clothing cuff of embodiment 45, wherein the re-sealable opening comprises a button. 52. The clothing cuff of embodiment 45, wherein the re-sealable opening comprises a belt. 53. The clothing cuff of embodiment 45, wherein the re-sealable opening comprises a hook and loop fastener. 54. The clothing cuff of embodiment 45, wherein the re-sealable opening comprises a magnetic closure. 55. The clothing cuff of embodiment 45, wherein opening the re-sealable opening releases tension in the interior material when deployed around a prosthetic. 56. The clothing cuff of embodiment 45, wherein the re-sealable opening comprises a drawstring. 57. The clothing cuff of embodiment 45, wherein the interior material has a width of at least 1 cm. 58. The clothing cuff of embodiment 45, wherein the interior material has a width of at least 2 cm. 59. The clothing cuff of embodiment 45, wherein the interior material has a width of at least 5 cm. 60. The clothing cuff of embodiment 45, wherein the interior material has a width of at least 10 cm.

61. The clothing cuff of embodiment 45, wherein the interior material comprises rubber. 62. The clothing cuff of embodiment 45, wherein the interior material comprises latex. 63. The clothing cuff of embodiment 45, wherein the interior material comprises silicone. 64. The clothing cuff of embodiment 45, wherein the interior material comprises elastomer. 65. The clothing cuff of embodiment 45, wherein the interior material comprises a beaded silicone band. 66. The clothing cuff of embodiment 45, wherein the interior material comprises polyurethane. 67. The clothing cuff of embodiment 45, wherein the interior material secures a prosthetic deployed within the cuff when the re-sealable opening is sealed. 68. The clothing cuff of any one of embodiments 45-67, configured for a below-knee pant leg opening. 69. The clothing cuff of any one of embodiments 45-67, configured for an above-knee pant leg opening. 70. The clothing cuff of any one of embodiments 45-67, configured for a below elbow shirt opening. 71. The clothing cuff of any one of embodiments 45-67, configured for an above elbow shirt opening. 72. An article of clothing comprising a clothing cuff of any one of embodiments 45-71, and further comprising a second cuff not comprising the interior material. 73. An article of clothing comprising at least two clothing cuffs of any one of embodiments 45-71.

74. Devices for securing a prosthetic limb comprising a. a cuff comprising a band material having an elastic deformability and an interior surface having a coefficient of friction that prevents sliding of an object interior to the clothing cuff across the interior surface; wherein the band material is configured to recoil to a diameter substantially less than a diameter of an adjacent portion of a clothing article; b. a re-sealable opening that bisects the band material; and c. a securing strap that tethers the cuff to the torso of a user. 75. The device of embodiment 74, configured to secure an above-elbow prosthetic. 76. The device of embodiment 74, configured to secure a below-elbow prosthetic. 77. The device of embodiment 74, configured to secure an above-knee prosthetic. 78. The device of embodiment 74, configured to secure a below-elbow prosthetic. 79. The device of embodiment 74, wherein the re-sealable opening comprises a zipper. 80. The device of embodiment 74, wherein the re-sealable opening comprises a snap. 81. The device of embodiment 74, wherein the re-sealable opening comprises a button. 82. The device of embodiment 74, wherein the re-sealable opening comprises a belt. 83. The device of embodiment 74, wherein the re-sealable opening comprises a hook and loop fastener. 84. The device of embodiment 74, wherein the re-sealable opening comprises a magnetic closure. 85. The device of embodiment 74, wherein opening the re-sealable opening releases tension in the interior material when deployed around a prosthetic. 86. The device of embodiment 74, wherein the re-sealable opening comprises a drawstring. 87. The device of embodiment 74, wherein the band material has a width of at least 1 cm. 88. The device of embodiment 74, wherein the band material has a width of at least 2 cm. 89. The device of embodiment 74, wherein the band material has a width of at least 5 cm. 90. The device of embodiment 74, wherein the band material has a width of at least 10 cm. 91. The device of embodiment 74, wherein the band material comprises rubber. 92. The device of embodiment 74, wherein the band material comprises latex. 93. The device of embodiment 74, wherein the band material comprises silicone. 94. The device of embodiment 74, wherein the band material comprises elastomer. 95. The device of embodiment 74, wherein the band material comprises a beaded silicone band. 96. The device of embodiment 74, wherein the band material comprises polyurethane. 97. The device of embodiment 74, wherein the band material secures a prosthetic deployed within the device when the re-sealable opening is sealed.

### EXAMPLES

The following illustrative examples are representative of embodiments of the composition, systems, and methods described herein and are not meant to be limiting in any way.

### Example 1. Adaptive Clothing for a Knee Prosthetic Limb

A pair of adaptive pants as described herein can be worn by an individual who had an amputation of at least one of their legs and wears a prosthetic limb on the amputated residual limb. The adaptive pants can have a body portion that fits around the waist of the individual and at least one pants leg with the access opening system as described herein to surround the residual limb with the prosthetic limb. The adaptive pants can have an access opening system comprising an access opening, a seal comprising a slip-resistant material that can be coupled circumferentially to the inner surface of the pants leg near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. The adaptive pants can provide stability for the prosthetic limb to stay on the residual limb and prevent undesired uncoupling of the prosthetic limb from the residual limb. The individual can access the prosthetic limb more discreetly and in public settings with the adaptive clothing with the access opening system to adjust the prosthetic limb.

### Example 2. Adaptive Clothing for an Arm Prosthetic Limb

An adaptive shirt as described herein can be worn by an individual who had an amputation of at least one of their arms and wears a prosthetic limb on the amputated residual limb. The adaptive shirt can have a body portion that fits around the torso of the individual and at least one sleeve with the access opening system as described herein to surround the residual limb with the prosthetic limb. The adaptive shirt can have an access opening system comprising an access opening, a seal comprising a slip-resistant material that can be coupled circumferentially to the inner surface of the sleeve near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. The adaptive shirt can provide stability for the prosthetic limb to stay on the residual limb and prevent undesired uncoupling of the prosthetic limb from the residual limb. The individual can access the prosthetic limb more discreetly and in public settings with the adaptive clothing with the access opening system to adjust the prosthetic limb.

### Example 3. An Access Opening System

A typical article of clothing can be modified to include an access opening system as described herein. The access opening system may comprise an access opening, a seal comprising a slip-resistant material that can be coupled circumferentially to the inner surface of the sleeve near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. The clothing adapted with the access opening system can provide stability for the prosthetic limb to stay on the residual limb and prevent undesired uncoupling of the prosthetic limb from the residual limb. The individual can access the prosthetic limb more discreetly and in public settings with the clothing adapted with the access opening system to adjust the prosthetic limb.

### Example 4. A Clothing Cuff

An article of clothing can have a clothing cuff that comprises an interior material, a re-sealable opening that bisects the interior material, and an exterior material comprising a clothing fabric. The interior material may have an elastic deformability and an interior surface having a coefficient of friction that prevents sliding of an object interior to the clothing cuff across the interior surface. The interior material may be a stretchy material that can return to its original dimensions without any force. The interior material may be a slip-resistant material that can help to hold a prosthetic limb in place on the residual limb. The interior material may recoil to a diameter substantially less than a diameter of an adjacent portion of the clothing. The clothing having such a cuff can provide stability for the prosthetic limb to stay on the residual limb and prevent undesired uncoupling of the prosthetic limb from the residual limb. The clothing allows the wearer to access the prosthetic limb more discreetly and in public settings to adjust the prosthetic limb. This can provide more comfort for the wearers of the clothing with the cuff.

### Example 5. A Device for Securing a Prosthetic Limb

An article of clothing can have a device for securing a prosthetic limb comprising a cuff, a re-sealable opening that bisects a band material of the cuff; and a securing strap that tethers the cuff to the torso of a user. The cuff may comprise a band material that is elastic and an interior surface having a coefficient of friction that prevents sliding of an object interior to the clothing cuff across the interior surface. The band material may be configured to recoil to a diameter substantially less than a diameter of an adjacent portion of a clothing article. The interior material may be a stretchy material that can return to its original dimensions without any force. The interior material may be a slip-resistant material that can help to hold a prosthetic limb in place on the residual limb. The clothing having such a device can provide stability for the prosthetic limb to stay on the residual limb and prevent undesired uncoupling of the prosthetic limb from the residual limb. The clothing allows the wearer to access the prosthetic limb more discreetly and in public settings to adjust the prosthetic limb. This can provide more comfort for the wearers of the clothing with the device.

### Example 6. Typical Clothing Worn by an Individual with a Leg Amputation

An individual with an amputation of one of their legs wears a prosthetic limb on the amputated residual limb. The individual wears a typical pair of pants with two pant legs that fall to the individual's ankle. As the individual walks and moves, the excess fabric in the pants leg around the prosthetic limb can bunch up and rub against the prosthetic limb, making it more likely for the pants leg to develop holes and for the pants to become damaged. In addition, as the individual moves, the individual may need to adjust the prosthetic limb. For example, a connection or a seal between the prosthetic limb and the residual limb can be broken, and the compromised connection or seal can cause discomfort for the individual that can be alleviated by adjusting the prosthetic limb. For the individual to adjust the prosthetic limb, the individual may remove the pants from the waist to the thigh to gain access to the residual limb and the prosthetic limb to adjust the prosthetic limb. The individual is not able to gain access to the residual limb and the prosthetic limb without removing a substantial portion of the pants and from the waist.

### Example 7. Adaptive Clothing Worn by an Individual with an Above the Knee Amputation

An individual with an amputation of one of their legs at above the knee wears a prosthetic limb on the amputated residual limb. The individual wears a pair of adaptive pants with one pant leg that fall to the individual's ankle and one pants leg with the access opening closure system as described herein to surround the residual limb with the prosthetic limb. As the individual walks and moves, there may not be excess fabric in the pants leg around the prosthetic limb that can bunch up or rub against the prosthetic limb. In addition, as the individual moves, the individual may need to adjust the prosthetic limb. For example, a connection or a seal between the prosthetic limb and the residual limb can be broken, and the compromised connection or seal can cause discomfort for the individual that can be alleviated by adjusting the prosthetic limb. For the individual to adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the adaptive clothing as well as when the individual adjusts the prosthetic limb while wearing the adaptive clothing. The individual can gain access to the prosthetic limb to adjust it without removing the pants from the waist or a substantial portion of the pants. The individual can access the prosthetic limb more discreetly and in public settings with the adaptive clothing with the access opening system. The adaptive pants can provide stability for the prosthetic limb to stay on the residual limb and prevent undesired compromised connection or seal of the prosthetic limb to the residual limb.

### Example 8. Adaptive Clothing Worn by an Individual with a Below the Knee Amputation

An individual with an amputation of one of their legs at below the knee wears a prosthetic limb on the amputated residual limb. The individual wears a pair of adaptive pants with one pant leg that fall to the individual's ankle and one pants leg with the access opening closure system as described herein to surround the residual limb with the prosthetic limb. As the individual walks and moves, there may not be excess fabric in the pants leg around the prosthetic limb that can bunch up or rub against the prosthetic limb. In addition, as the individual moves, the individual may need to adjust the prosthetic limb. For example, a connection or a seal between the prosthetic limb and the residual limb can be broken, and the compromised connection or seal can cause discomfort for the individual that can be alleviated by adjusting the prosthetic limb. For the individual to adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the adaptive clothing as well as when the individual adjusts the prosthetic limb while wearing the adaptive clothing. The individual can gain access to the prosthetic limb to adjust it without removing the pants from the waist or a substantial portion of the pants. The individual can access the prosthetic limb more discreetly and in public settings with the adaptive clothing with the access opening system. The adaptive pants can provide stability for the prosthetic limb to stay on the residual limb and prevent undesired compromised connection or seal of the prosthetic limb to the residual limb.

### Example 9. Typical Clothing Worn by an Individual with an Arm Amputation

An individual with an amputation of one of their arms wears a prosthetic limb on the amputated residual limb. The individual wears a typical shirt with two sleeves that fall to the individual's wrist. As the individual moves and goes about their daily activity, the excess fabric in the sleeve around the prosthetic limb can bunch up and rub against the prosthetic limb, making it more likely for the sleeve to develop holes and for the shirt to become damaged. In addition, the individual may need to adjust the prosthetic limb. For example, a connection or a seal between the prosthetic limb and the residual limb can be broken, and the compromised connection or seal can cause discomfort for the individual that can be alleviated by adjusting the prosthetic limb. For the individual to adjust the prosthetic limb, the individual may remove the shirt to gain access to the residual limb and the prosthetic limb to adjust the prosthetic limb. The individual is not able to gain access to the residual limb and the prosthetic limb without removing a substantial portion of the shirt from the torso.

### Example 10. Adaptive Clothing Worn by an Individual with an Above the Elbow Amputation

An individual with an amputation of one of their arms at above the elbow wears a prosthetic limb on the amputated residual limb. The individual wears an adaptive shirt with one sleeve that fall to the individual's wrist and one sleeve with the access opening closure system as described herein to surround the residual limb with the prosthetic limb. As the individual moves, there may not be excess fabric in the sleeve around the prosthetic limb that can bunch up or rub against the prosthetic limb. In addition, as the individual moves, the individual may need to adjust the prosthetic limb. For example, a connection or a seal between the prosthetic limb and the residual limb can be broken, and the compromised connection or seal can cause discomfort for the individual that can be alleviated by adjusting the prosthetic limb. For the individual to adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the adaptive clothing as well as when the individual adjusts the prosthetic limb while wearing the adaptive clothing. The individual can gain access to the prosthetic limb to adjust it without removing the shirt or a substantial portion of the shirt and exposing the shoulder and the upper arm. The individual can access the prosthetic limb more discreetly and in public settings with the adaptive clothing with the access opening system. The adaptive shirt can provide stability for the prosthetic limb to stay on the residual limb and prevent undesired compromised connection or seal of the prosthetic limb to the residual limb.

### Example 11. Adaptive Clothing Worn by an Individual with a Below the Elbow Amputation

An individual with an amputation of one of their arms at below the elbow wears a prosthetic limb on the amputated residual limb. The individual wears an adaptive shirt with one sleeve that falls to the individual's wrist and one sleeve with the access opening closure system as described herein to surround the residual limb with the prosthetic limb. As the individual moves, there may not be excess fabric in the sleeve around the prosthetic limb that can bunch up or rub against the prosthetic limb. In addition, as the individual moves, the individual may need to adjust the prosthetic limb. For example, a connection or a seal between the prosthetic limb and the residual limb can be broken, and the compromised connection or seal can cause discomfort for the individual that can be alleviated by adjusting the prosthetic limb. For the individual to adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the adaptive clothing as well as when the individual adjusts the prosthetic limb while wearing the adaptive clothing. The individual can gain access to the prosthetic limb to adjust it without remove the shirt or a substantial portion of the shirt and exposing the shoulder and the upper arm. The individual can access the prosthetic limb more discreetly and in public settings with the adaptive clothing with the access opening system. The adaptive shirt can provide stability for the prosthetic limb to stay on the residual limb and prevent undesired compromised connection or seal of the prosthetic limb to the residual limb.

### Example 12. Amputee Suspension Sleeve Worn by an Individual with an Above the Knee Amputation

An individual with an amputation of one of their legs at above the knee wears a prosthetic limb on the amputated residual limb. The individual wears an amputee suspension sleeve with one sleeve with the access opening closure system as described herein for the residual limb with the prosthetic limb and a torso portion that fits at least partially around the waist. For the individual to adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the amputee suspension sleeve as well as when the individual adjusts the prosthetic limb while wearing the amputee suspension sleeve. The amputee suspension sleeve can provide stability for the prosthetic limb to stay on the residual limb and prevent undesired compromised connection or seal of the prosthetic limb to the residual limb. The individual can gain access to the prosthetic limb to adjust it. The individual can access the prosthetic limb more discreetly and in public settings with the amputee suspension sleeve.

### Example 13. Amputee Suspension Sleeve Worn by an Individual with an Above the Elbow Amputation

An individual with an amputation of one of their arms at above the elbow wears a prosthetic limb on the amputated residual limb. The individual wears an amputee suspension sleeve with one sleeve with the access opening closure system as described herein to surround the residual limb with the prosthetic limb and a torso portion that fits from the shoulder with the residual limb across the upper chest to the under arm of the other shoulder. For the individual to adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the amputee suspension sleeve as well as when the individual adjusts the prosthetic limb while wearing the amputee suspension sleeve. The amputee suspension sleeve can provide stability for the prosthetic limb to stay on the residual limb and prevent undesired compromised connection or seal of the prosthetic limb to the residual limb. The individual can gain access to the prosthetic limb to adjust it. The individual can access the prosthetic limb more discreetly and in public settings with the amputee suspension sleeve.

### Example 14. Hybrid Pants with an Amputee Suspension Sleeve for an Above the Knee Amputation

An individual with an amputation of one of their legs at above the knee wears a prosthetic limb on the amputated residual limb. The individual wears a pair of hybrid pants integrated with an amputee suspension sleeve. The amputee suspension sleeve can have one sleeve with the access opening closure system as described herein to surround the residual limb with the prosthetic limb and a torso portion that fits at least partially around the waist. The pants can comprise a material, common for a typical pair of pants, and the amputee suspension sleeve can comprise a second material. For the individual to adjust the prosthetic limb, the individual may open up the access opening on the amputee suspension sleeve by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the hybrid pants as well as when the individual adjusts the prosthetic limb while wearing the hybrid pants. The hybrid pants can provide stability for the prosthetic limb to stay on the residual limb and prevent undesired compromised connection or seal of the prosthetic limb to the residual limb. The individual can gain access to the prosthetic limb to adjust it without removing the hybrid pants from the waist or a substantial portion of the hybrid pants. The individual can access the prosthetic limb more discreetly and in public settings with the hybrid pants.

### Example 15. Hybrid Amputee Suspension Sleeve for an Above the Elbow Amputation

An individual with an amputation of one of their arms at above the elbow wears a prosthetic limb on the amputated residual limb. The individual wears a hybrid shirt integrated with an amputee suspension sleeve. The amputee suspension sleeve can have one sleeve with the access opening closure system as described herein for the residual limb with the prosthetic limb and a torso portion that fits from the shoulder with the residual limb across the upper chest to the under arm of the other shoulder. The shirt can comprise a material, common for a typical shirt, and the amputee suspension sleeve can comprise a second material. For the individual to adjust the prosthetic limb, the individual may open up the access opening of the hybrid shirt by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the hybrid shirt as well as when the individual adjusts the prosthetic limb while wearing the hybrid shirt. The hybrid shirt can provide stability for the prosthetic limb to stay on the residual limb and prevent undesired compromised connection or seal of the prosthetic limb to the residual limb. The individual can gain access to the prosthetic limb to adjust it without removing the hybrid shirt or a substantial portion of the hybrid shirt to expose the shoulder and the residual limb. The individual can access the prosthetic limb more discreetly and in public settings with the hybrid shirt.

### Example 16. Adapting the Access Opening System to Typical Pants

A typical article of clothing can be modified to include an access opening system as described herein. For example, a pair of pants can be modified to include the access opening system by cutting at least one pants leg to a shorter length that can accommodate to the access opening system at a desired length. The desired length would be where the access opening system can provide a quick access to a prosthetic limb without removing the pants or a substantial portion of the pants. The access opening system can be adhered to the shortened pants leg by sewing, fabric tape, or other commonly used methods to couple pieces of fabric together. The modified pants with the access opening system can provide its wearer the capability to gain access to the prosthetic limb to adjust it without removing the pants from the waist or a substantial portion of the pants. To adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the pants adapted with an access opening system as well as when the individual adjusts the prosthetic limb while wearing the adapted pants. The adapted pants can provide stability for the prosthetic limb to stay on the residual limb and prevent undesired compromised connection or seal of the prosthetic limb to the residual limb.

### Example 17. Adapting the Access Opening System to Typical Shirt

A typical article of clothing can be modified to include an access opening system as described herein. For example, a shirt can be modified to include the access opening system by cutting at least one sleeve to a shorter length that can accommodate to the access opening system at a desired length. The desired length would be where the access opening system can provide a quick access to a prosthetic limb without removing the shirt or a substantial portion of the shirt. The access opening system can be adhered to the shortened sleeve by sewing, fabric tape, or other commonly used methods to couple pieces of fabric together. The modified shirt with the access opening system can provide its wearer the capability to gain access to the prosthetic limb to adjust it without removing the shirt to expose the shoulder and the upper arm or a substantial portion of the shirt. To adjust the prosthetic limb, the individual may open up the access opening by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed when the individual first wears the shirt adapted with an access opening system as well as when the individual adjusts the prosthetic limb while wearing the adapted shirt. The adapted shirt can provide stability for the prosthetic limb to stay on the residual limb and prevent undesired compromised connection or seal of the prosthetic limb to the residual limb.

### Example 18. Adaptive Clothing for a Double Amputee

A pair of adaptive pants as described herein can be worn by an individual who had an amputation of both of their legs and wears a prosthetic limb on each of the amputated residual limbs. The adaptive pants can have a body portion that fits around the waist of the individual and two pants legs, each pants leg with an access opening system as described herein to surround the residual limb with the prosthetic limb. The adaptive pants can have an access opening system comprising an access opening, a seal comprising a slip-resistant material that can be coupled circumferentially to the inner surface of the pants leg near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. When both of the amputations are at above the knee or below the knee, both access openings on the adaptive pants can be located at similar lengths along the pants leg. The adaptive pants can provide stability for the prosthetic limbs to stay on the residual limbs and prevent undesired uncoupling of the prosthetic limbs from the residual limbs. The individual can access the prosthetic limbs more discreetly and in public settings with the adaptive clothing with the access opening system to adjust the prosthetic limbs.

### Example 19. Adaptive Clothing for a Double Amputee

A pair of adaptive pants as described herein can be worn by an individual who had an amputation of both of their legs and wears a prosthetic limb on each of the amputated residual limbs. The adaptive pants can have a body portion that fits around the waist of the individual and two pants legs, each pants leg with an access opening system as described herein to surround the residual limb with the prosthetic limb. The adaptive pants can have an access opening system comprising an access opening, a seal comprising a slip-resistant material that can be coupled circumferentially to the inner surface of the pants leg near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. When one amputation is above the knee and another amputation is below the knee, the access openings on the adaptive pants can be located at different lengths along the pants leg that correspond to the location of the socket of the prosthetic limb. The access opening to access the residual limb for the below the knee amputation can be placed below the knee and lower than the access opening to access the residual limb for the above the knee amputation, which can be placed above the knee of the individual wearing the pants. The adaptive pants can provide stability for the prosthetic limbs to stay on the residual limbs and prevent undesired uncoupling of the prosthetic limbs from the residual limbs. The individual can access the prosthetic limbs more discreetly and in public settings with the adaptive clothing with the access opening system to adjust the prosthetic limbs.

### Example 20. Adaptive Clothing for a Double Amputee

An adaptive shirt as described herein can be worn by an individual who had an amputation of both of their arms and wears a prosthetic limb on each of the amputated residual limbs. The adaptive shirt can have a body portion that fits around the torso of the individual and two arm sleeves, each sleeve with an access opening system as described herein to surround the residual limb with the prosthetic limb. The adaptive shirt can have an access opening system comprising an access opening, a seal comprising a slip-resistant material that can be coupled circumferentially to the inner surface of the sleeve near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. When both of the amputations are at above the elbow or below the elbow, both access openings on the adaptive shirt can be located at similar lengths along the sleeve. The adaptive shirt can provide stability for the prosthetic limbs to stay on the residual limbs and prevent undesired uncoupling of the prosthetic limbs from the residual limbs. The individual can access the prosthetic limbs more discreetly and in public settings with the adaptive shirt with the access opening system to adjust the prosthetic limbs.

### Example 21. Adaptive Clothing for a Double Amputee

An adaptive shirt as described herein can be worn by an individual who had an amputation of both of their arms and wears a prosthetic limb on each of the amputated residual limbs. The adaptive shirt can have a body portion that fits around the torso of the individual and two arm sleeves, each sleeve with an access opening system as described herein to surround the residual limb with the prosthetic limb. The adaptive shirt can have an access opening system comprising an access opening, a seal comprising a slip-resistant material that can be coupled circumferentially to the inner surface of the sleeve near the access opening, and a closure for the access opening. The closure can be at the edge of the access opening and bisect the circumference of the seal. The closure can be adjusted to increase or decrease the overlapping area of the access opening and, in turn, adjust the circumference of the seal. The seal and/or closure can be adjusted to change the tightness of the seal around the prosthetic limb. When one amputation is above the elbow and another amputation is below the elbow, the access openings on the adaptive shirt can be located at different lengths along the sleeve that correspond to the location of the socket of the prosthetic limb. The access opening to access the residual limb for the below the elbow amputation can be placed below the elbow and lower than the access opening to access the residual limb for the above the elbow amputation, which can be placed above the elbow of the individual wearing the shirt. The adaptive shirt can provide stability for the prosthetic limbs to stay on the residual limbs and prevent undesired uncoupling of the prosthetic limbs from the residual limbs. The individual can access the prosthetic limbs more discreetly and in public settings with the adaptive shirt with the access opening system to adjust the prosthetic limbs.

### Example 22. Adaptive Clothing Worn by an Individual with Double Leg Amputations

An individual with amputations of both of their legs wears a prosthetic limb on each of the amputated residual limbs. The individual wears a pair of adaptive pants with two pants legs, each pants leg with an access opening closure system as described herein to surround each residual limb with the prosthetic limb. For the individual to adjust at least one of the prosthetic limbs, the individual may open up at least one of the access openings by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed as the individual first wears the adaptive clothing as well. The individual can gain access to the prosthetic limb to adjust it without removing the pants from the waist or a substantial portion of the pants. The adaptive pants can provide stability for the prosthetic limb to stay on the residual limb and prevent undesired compromised connection or seal of the prosthetic limb to the residual limb.

### Example 23. Adaptive Clothing Worn by an Individual with Double Arm Amputations

An individual with amputations of both of their arms wears a prosthetic limb on each of the amputated residual limbs. The individual wears an adaptive shirt with two sleeves, each sleeve with an access opening closure system as described herein to surround each residual limb with the prosthetic limb. For the individual to adjust at least one of the prosthetic limbs, the individual may open up at least one of the access openings by opening the closure and loosening the seal to gain access to the residual limb and the prosthetic limb and to adjust the prosthetic limb. Once the prosthetic limb has been adjusted, the seal can be placed back on the prosthetic limb and the closure can be fastened to close the access opening. The seal can be further tightened to secure the seal on the socket of the prosthetic limb. The steps involved in closing the access opening may be performed as the individual first wears the adaptive clothing as well. The individual can gain access to the prosthetic limb to adjust it without removing the shirt or a substantial portion of the shirt to expose their shoulder. The adaptive shirt can provide stability for the prosthetic limb to stay on the residual limb and prevent undesired compromised connection or seal of the prosthetic limb to the residual limb.

### Example 24. An Access Opening System

An access opening system as described herein comprises an access opening, a seal comprising a slip-resistant material that is coupled circumferentially to the inner surface of a sleeve near the access opening, and a closure for the access opening. A number of devices or mechanisms for closure resealing are contemplated herein. For example, in some embodiments the closure comprises a zipper and the seal comprises at least one strip of silicone, but other devices for resealing of the closure and seals are also consistent with the disclosure herein.

### Example 25. A Clothing Cuff

An article of clothing has a clothing cuff that comprises an interior material, a re-sealable opening that bisects the interior material, and an exterior material comprising a clothing fabric. The interior material is at least one strip of silicone that prevents sliding of an object interior to the clothing cuff across the interior surface. A number of alternatives for re-sealable openings are consistent with the disclosure herein. Some re-sealable openings comprise a zipper. However, a number of alternative re-sealable openings are also contemplated.

### Example 26. A Device for Securing a Prosthetic Limb

An article of clothing has a device for securing a prosthetic limb comprising a cuff, a re-sealable opening that bisects a band material of the cuff; and a securing strap that tethers the cuff to the torso of a user. A number of alternatives for re-sealable openings are consistent with the disclosure herein. The re-sealable opening comprises a zipper and the band material comprises at least one strip of silicone. In some instances, the band material is elastic and an interior surface has a coefficient of friction that prevents sliding of an object interior to the clothing cuff across the interior surface. However, a number of alternative re-sealable openings and band materials are also contemplated.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. An article of clothing comprising:
a. a body portion configured to at least partially surround a torso of an individual;
b. a first limb portion having an outer surface and an inner surface, and a proximal end portion connected to the body portion and a distal end portion having a first opening at a distal end and configured to at least partially surround a limb of the individual;
c. an access opening on the distal end portion proximal to the first opening;
d. a seal coupled circumferentially to the inner surface at a distal end portion of the access opening and comprising a slip resistant material; and
e. a closure coupled to edge portions of the access opening, wherein a distal end of the closure meets or disrupts a circumference of the seal and wherein the closure is configured to open and close the access opening and configured to adjust the circumference of the seal by closing the closure to adjust an overlapping area of the edge portions of the access opening.

2. The article of clothing of claim 1, wherein the access opening is configured to be located at one of above a knee, below the knee, above an elbow, and below the elbow when the article is worn by the individual.

3. The article of clothing of claim 1, wherein the seal is configured to fit around and form a tight seal to an outer surface of a proximal end portion of a prosthetic limb when the prosthetic limb is fitted to the limb of the individual.

4. The article of clothing of claim 3, wherein the seal is configured to secure the prosthetic limb from rotating around a longitudinal axis of the limb or translating off the axis.

5. The article of clothing of claim 3, wherein the access opening is configured to provide access to the prosthetic limb without removing a substantial portion of the article from the individual by opening the closure.

6. (canceled)

7. The article of clothing of claim 1, wherein the article comprises a flexible fabric.

8. (canceled)

9. The article of clothing of claim 1, wherein the flexible fabric is configured to support a high range of motion of the limb and of the prosthetic limb and does not catch on the prosthetic limb.

10. The article of clothing of claim 1, wherein the closure is configured to close the access opening.

11. The article of clothing of claim 10, wherein the closure comprises a zipper, a hook and loop fastener (Velcro), a drawstring, a belt, an elastic band, a plurality of buttons, a plurality of snap closures, a buckle, a magnetic closure, or a combination of a rope and a plurality of holes or hooks.

12. (canceled)

13. (canceled)

14. The article of clothing of claim 1, wherein the seal comprises a material with a high coefficient of friction and a high elastic modulus.

15. The article of clothing of claim 14, wherein the material has a coefficient of friction equal to or greater than about 1.0.

16. (canceled)

17. The article of clothing of claim 14, wherein the seal comprises a textured surface, wherein the textured surface provides slip resistance.

18. (canceled)

19. (canceled)

20. The article of clothing of claim 14, wherein the material partially covers the circumference of the seal.

21. The article of clothing of claim 3, wherein the first limb portion comprises a durable portion, wherein the durable portion is coupled to the first limb portion near the access opening and covers at least a portion of the prosthetic limb.

22. (canceled)

23. (canceled)

24. (canceled)

25. (canceled)

26. (canceled)

27. (canceled)

28. (canceled)

29. (canceled)

30. (canceled)

31. (canceled)

32. The article of clothing of claim 1, wherein the article comprises a second limb portion, the second limb portion having a second outer surface and an inner surface, and a proximal end portion connected to the body portion and a distal end portion having a second opening at a distal end and configured to at least partially surround a second limb of the individual.

33. The article of clothing of claim 32, wherein the second limb portion comprises
a. a second access opening on the distal end portion proximal to the second opening;
b. a second seal coupled circumferentially to the inner surface at a distal end portion of the second access opening and comprising a slip resistant material; and
c. a second closure at edge portions of the second access opening, wherein a distal end of the closure meets or disrupts a circumference of the second seal and wherein the closure is configured to open and close the access opening and configured to adjust the circumference of the second seal by closing the closure to adjust an overlapping area of the edge portions of the second access opening.

34. The article of clothing of claim 32, wherein the second limb portion is same length as the first limb portion.

35. (canceled)

36. (canceled)

37. The article of clothing of claim 1, wherein the distal end of the first limb portion extends distally further than the access opening.

38. (canceled)

39. (canceled)

40. (canceled)

41. An access opening assembly on an article of clothing, the access opening assembly comprising:
a. an access opening on a distal end portion of a body portion of an article of clothing and proximal to a distal opening of the body portion;
b. a seal comprising a slip resistant material and coupled circumferentially to an inner surface of the body portion at a distal end portion of the access opening; and
c. a closure at edge portions of the access opening, wherein a distal end of the closure meets or disrupts a circumference of the seal and wherein the closure is the closure is configured to open and close the access opening and configured to adjust the circumference of the seal by closing the closure to adjust an overlapping area of the edge portions of the access opening.

42. (canceled)

43. A clothing cuff comprising
a. an interior material having
an elastic deformability and
an interior surface having a coefficient of friction that does not support sliding of an object interior to the clothing cuff across the interior surface;
wherein the interior material is configured to recoil to a diameter substantially less than a diameter of an adjacent portion of a clothing article;
b. a re-sealable opening that bisects the interior material; and
c. an exterior material comprising a clothing fabric.

44.-94. (canceled)
